(19) United States
(12) Patent Application Publication
(10) Pub. No.: US 2020/0061388 A1
(43) Pub. Date: Feb. 27, 2020

(54) SYSTEMS, METHODS, AND FLEXIBLE OPTICAL WAVEGUIDES FOR SCLERAL CROSSLINKING

(71) Applicant: The General Hospital Corporation, Boston, MA (US)

(72) Inventors: Seok Hyun Yun, Cambridge, MA (US); Sheldon J.J. Kwok, Cambridge, MA (US); Moonseok Kim, Cambridge, MA (US)

(21) Appl. No.: 16/463,925

(22) PCT Filed: Nov. 27, 2017
(86) PCT No.: PCT/US17/63245

§ 371 Date: May 24, 2019

Related U.S. Application Data
(60) Provisional application No. 62/426,906, filed on Nov. 28, 2016.

(51) Int. Cl. A61N 5/06; F21V 8/00

(52) U.S. Cl. CPC A61N 5/0613; G02B 6/0006; A61N 2005/067; A61N 2005/063; A61N 2005/0648; G02B 6/001

(57) ABSTRACT

A system for delivering light to a curved surface of a tissue of a subject. The system includes a light source; an optical coupler coupled to the light source; and a flexible waveguide coupled to the optical coupler. The waveguide has a first end and a second end with an elongated flat portion therebetween. Light from the light source is emitted substantially uniformly from along the elongated flat portion of the flexible waveguide, thereby delivering light substantially uniformly along the curved surface of the tissue.

### CROSS-REFERENCE TO RELATED APPLICATIONS

This application claims the benefit of U.S. Provisional Patent Application Ser. No. 62/426,906 filed on Nov. 28, 2016, and entitled “Flexible Optical Waveguide for Scleral Crosslinking and Myopia Control,” which is incorporated by reference herein in its entirety.

### FEDERAL FUNDING NOTICE

This invention was made with government support under NIBIB 2P41-EB015903, subproject ID: #7931, TRD1: QUANTITATIVE BIOMECHANICS IMAGING (PI: Seok-Hyun Andy Yun) awarded by the National Institutes of Health. The government has certain rights in the invention.

### BACKGROUND OF THE INVENTION

Myopia, or short-sightedness, is a rapidly growing disorder that could affect 2.5 billion people by 2020. Far beyond a mild inconvenience, myopia increases the risk of serious disorders such as retinal detachment, glaucoma, and cataracts, and is a leading cause of blindness worldwide. The global economic burden of myopia is immense, including consequences of uncorrected refractive error, costs of treatment, and need for long-term management. Since myopia severity increases with earlier onset of myopia, prophylactic strategies to prevent myopia progression are of great interest. Biomechanical weakening of the sclera (i.e. the white, protective layer of the eye) resulting in accelerated axial eye growth has been identified as a major cause of myopia. Sclera buckling surgery is a proposed technique for mechanically reinforcing the sclera, however this procedure is highly invasive and carries a high risk of complications.

### SUMMARY OF THE INVENTION

A promising and less invasive strategy for myopia control is photochemical collagen crosslinking, which uses a combination of photosensitizers and light. This technique is already commonly used in the clinic to strengthen the cornea to treat corneal ectasia. However, unlike corneal crosslinking (CXL), technical constraints have limited translation of scleral crosslinking (SXL), including:
The sclera is anatomically difficult to access, with soft tissues surrounding it in the eye socket, making the sclera difficult to irradiate with external laser illumination. A light delivery device must be flexible enough to wrap around the eyeball, while being thin enough (<2 mm) to safely insert underneath the rectus muscles.
Homogenous light delivery around the eyeball is a requirement. Uniform crosslinking around the eye is needed to ensure proper arrest of eye growth and prevent development of astigmatisms.
Care must be taken to avoid undesirable damage to the cells and blood vessels in the choroid, retina, and Tenon's capsule, as well as sclera. For example, any heat generation by active optical devices (e.g. light-emitting diodes) or photodamage by stray laser light should be minimized.

To address the above technical challenges, provided herein are embodiments of a flexible, polymer waveguides optimized for efficient and uniform delivery of light into biological tissues. In the disclosed design, a fiber-coupled laser source is inserted into the waveguide, which may then be wrapped around the eyeball to perform SXL. Discussed herein are strategies for designing waveguides with uniform light extraction, using a theoretical model for waveguide loss. Using flexible polymer-based waveguides, successful SXL-induced stiffening of the sclera around the equator of fresh porcine eyeballs is demonstrated.

In accordance with one aspect of the present disclosure, a system is provided for delivering light to a curved surface of a tissue of a subject. The system includes a light source; an optical coupler coupled to the light source; and a flexible waveguide coupled to the optical coupler. The waveguide has a first end and a second end with an elongated flat portion therebetween. Light from the light source is emitted substantially uniformly from along the elongated flat portion of the flexible waveguide, thereby delivering light substantially uniformly along the curved surface of the tissue.

In accordance with another aspect of the present disclosure, a method is provided for delivering light to a tissue of a subject. The method includes steps of: providing a light source coupled to a flexible waveguide by an optical coupler, the waveguide having a first end and a second end with an elongated flat portion therebetween; contacting the tissue with the waveguide; and directing light into the waveguide, the light traveling from the light source through the optical coupler into the waveguide, at least a portion of the light exiting the waveguide toward the tissue.

In accordance with yet another aspect of the present disclosure, an apparatus is provided. The apparatus includes a flexible waveguide having first end and a second end with an elongated flat portion therebetween.

In accordance with still another aspect of the present disclosure, a method is provided for treating myopia in a subject. The method includes steps of: contacting scleral tissue of the subject with a flexible waveguide, the waveguide having a first end and a second end with an elongated flat portion therebetween; and directing light into the waveguide with a light source, the light source coupled to the flexible waveguide by an optical coupler, the light traveling from the light source through the optical coupler into the waveguide, at least a portion of the light exiting the waveguide toward the tissue and interacting with a photosensitizer in the scleral tissue.

In accordance with one aspect of the present disclosure, a system is provided for delivering light to a tissue of a subject. The system includes a light source, an optical coupler coupled to the light source, and a flexible waveguide coupled to the optical coupler. The flexible waveguide has a first end and a second end with an elongated flat portion therebetween. The first end has a first thickness and the second end has a second thickness, where the first thickness is greater than the second thickness.

The foregoing and other aspects and advantages of the invention will appear from the following description. In the description, reference is made to the accompanying drawings which form a part hereof, and in which there is shown by way of illustration preferred embodiments of the invention. Such embodiments do not necessarily represent the full scope of the invention, however, and reference is made therefore to the claims herein for interpreting the scope of the invention.

### BRIEF DESCRIPTION OF THE DRAWINGS

FIG. 1 shows a schematic showing a total internal reflection-based waveguide. Input light is delivered by an optical fiber (blue line) to the slab waveguide made of materials with an effective refractive index, n_{wg}. Optical rays within the critical angle, θ_{c}, are guided via total internal reflection. Yet, optical scattering in the waveguide material and at the waveguide-tissue interface causes the guided light to be extracted into tissue (red arrows).

FIG. 2a shows images of a flexible waveguide according to one construction. A pigtail fiber (with NA=0.22) was used to couple light into the waveguide. FIG. 2b shows transmittance of the waveguide in the visible wavelengths.

FIG. 3 shows a schematic of a slab waveguide wrapped around an eyeball. t, thickness of the waveguide; R, radius of the eyeball; θ, incident angle of an optical ray (black lines) with respect to the normal axis of the outer surface, for which the optical beam is incident to the inner surface at a zero-shallow angle.

FIGS. 4a-4d show various multi-layer, core-cladded waveguide designs to block light leakage and enable efficient and safe light delivery in tissue environments. FIGS. 4a and 4b include an air spacer layer to ensure total internal reflection at the interface between the air and the outside of the waveguide. FIGS. 4c and 4d include the use of core containing a high refractive index (RI) material surrounded by a cladding of relatively low refractive index materials.

FIGS. 5a-5c show images of a construction of a core-cladded waveguide with a reflective layer. The waveguide includes a polyurethane core with a PDMS cladding and a thin sheet of reflective Mylar bonded to the outer surface. This cladded waveguide design enables efficient light delivery in tissue environments and prevents leakage of light through the outer surface.

FIG. 6a shows a schematic of a tapered waveguide showing less reflections near the proximal end and more reflections near the distal end, compared to a waveguide of constant thickness. FIG. 6b shows measurement of fluorescence intensity around a riboflavin-stained eyeball. FIG. 6c shows fluorescence intensity as a function of position for flat (lower trace, blue dots) and tapered (upper trace, red dots) waveguides; fitting of light extraction profile for flat waveguides to Eq. 4 is shown by the dotted blue line; predicted light extraction profile for tapered waveguides using Eq. 13 is shown by the dotted red line. Tapered waveguides show significantly more homogenous light extraction compared to flat waveguides with the same mean thickness.

FIG. 7a shows a schematic of a light coupling system. FIG. 7b shows a construction in which more than one pigtail optical fibers may be attached to a single waveguide at different locations to achieve a desired light profile. FIG. 7c shows a schematic of a light delivery and monitoring system. The excitation fight remaining in the waveguide after propagating through the waveguide length is monitored by a detector. At the same time, the fluorescence light that is generated from the photochemical agent (e.g. riboflavin) in the tissue that is captured and guided by the waveguide can also be measured.

FIG. 8 is a flow chart of an example process for delivering light to a tissue of a subject.

FIG. 9 is a flow chart of an example process of treating myopia in a subject.

FIG. 10a shows images of porcine eyeballs during (top) and after SXL (bottom) was performed using blue light; FIG. 10a (bottom image) shows a pattern of bleached riboflavin after 30 min irradiation. FIG. 10b shows photobleaching of riboflavin using mean intensities of 25-100 mW/cm².

FIG. 11a shows a stress-strain curve obtained from tensiometry comparing untreated sclera (green circles, lower trace, ‘control’) and crosslinked sclera with elastomer waveguide (light blue circles, upper trace, ‘with elastomer waveguide’) and direct illumination (purple squares, upper trace, ‘direct illumination’). At least six porcine eyes were used for each group. FIG. 11b shows Young's modulus at 8% strain for the three cases; SXL was conducted using 50 mW/cm² for 30 minutes. The stiffness of the scleral tissues at the proximally (dark blue) and distally (light blue) treated areas is not significantly different from each other and is similar to the positive control tissues (purple) irradiated by direct illumination without the waveguide.

### DETAILED DESCRIPTION

Provided are systems, methods, and apparatus for delivering light to tissue via a waveguide for therapeutic and other purposes. Light from a light source propagates through the waveguide by internal reflection and a portion of the light exits the waveguide and may impinge on a surrounding tissue with which the waveguide is in contact. The light impinging on the tissue may have therapeutic effects on the tissue, for example by interacting with a photosensitizer in the tissue. Light that travels through and is emitted from an end of the waveguide (e.g. excitation light from the light source or fluorescence light, for example from the tissue) can be detected and used to monitor progress of a therapeutic or other procedure.

Waveguide fabrication and optical characterization

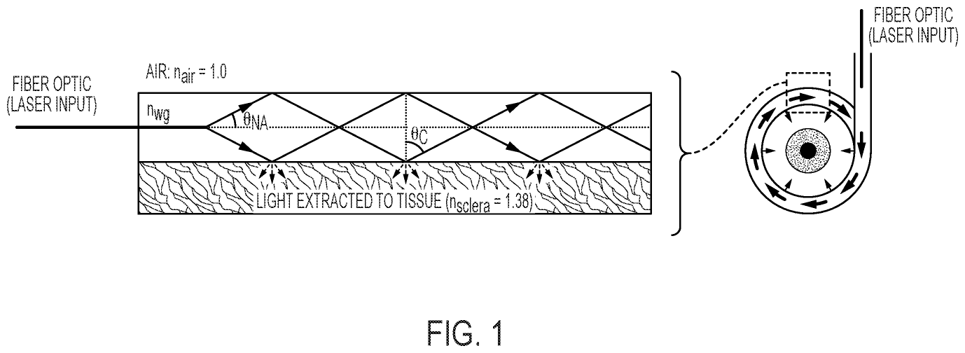

To achieve efficient light propagation inside the waveguide (FIG. 1), the waveguide in some embodiments has an effective refractive index above that of tissue (in this case n_{sclera}=1.38). In one experiment, a flexible polymer (elastomer), consisting of polydimethysiloxane (PDMS), which has a refractive index of ˜1.42 was used to create a waveguide. An input fiber with a numerical aperture (NA=0.22) corresponding to incident angles, θ_{NA}, above the critical angle, enabling total internal reflection, was used to couple light into the waveguide.

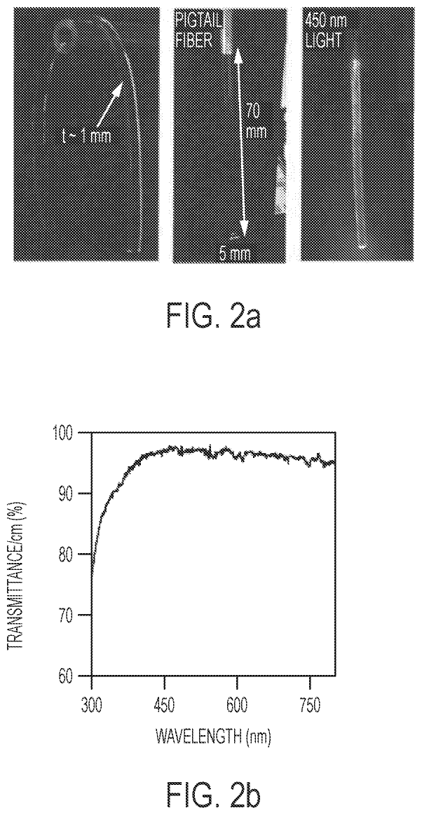

An image of an embodiment of the elastomer waveguide is shown in FIG. 2a. The dimensions in one particular embodiment of the waveguides are 70 mm×5 mm, with a thickness of approximately 1 mm. In various embodiments the waveguides are highly transparent in the visible and near infrared wavelengths (e.g. 400-800 nm), having above 95% transmittance per cm (FIG. 2b). This high transparency is well suited for applications such as SXL. On the other hand, in various embodiments optical absorption by the waveguide material should be as little as possible to minimize absorption-induced heat generation.

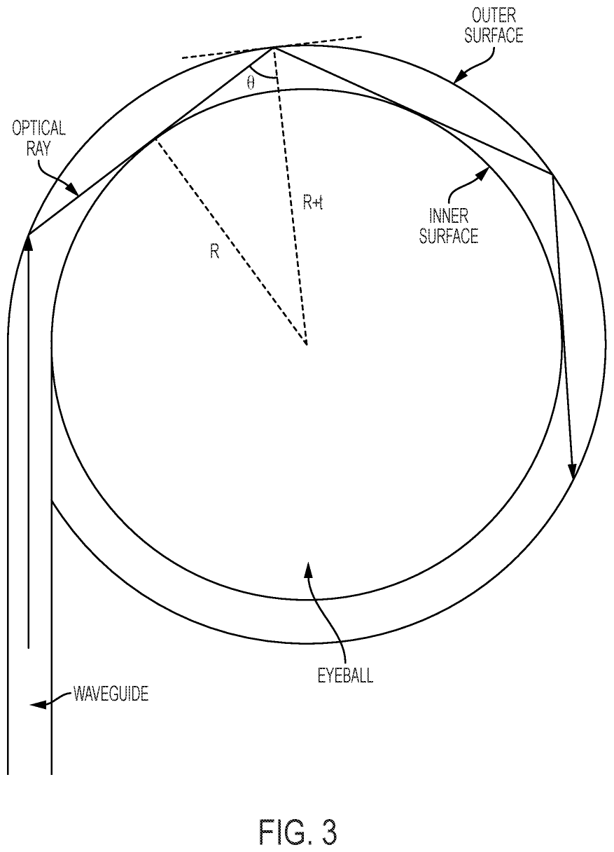

When the slab waveguide is wrapped around a structure such as an eyeball, the internal reflection angles are generally altered, for example due to the curvature of the waveguide due to being wrapped around the structure. This situation is illustrated in FIG. 3, in which a representative path of a beam that enters the curved part of the waveguide from the straight part of the waveguide along the axis is depicted. The central optical ray meets the curved outer surface of the waveguide at an angle given by:

$$θ = {sin}^{- 1} ( \frac{R}{R + t} ) \qquad ( 1 )$$

For example, for a waveguide having a thickness t=1 mm and being wrapped around a structure which gives a radius of curvature of R=12 mm, the angle is θ=˜67.4 deg. For a waveguide made of a material such as PDMS, which has an index of refraction of n=1.42, the critical angle for total internal reflection at the PDMS-air interface (i.e. the outer-facing side of the waveguide, which in this instance is not in contact with a structure such as the sclera) is 44.8 deg. Therefore, a PDMS-based waveguide such as that described above and shown in FIG. 1 can guide the optical ray via total internal reflection. At the inner surface, which is in contact with tissue (e.g. sclera), the incident angle is near 90 deg.; therefore, although the critical angle is much higher, ˜76.3 deg., total internal reflection is supported.

In clinical applications, however, the waveguide may be inserted into the extraocular socket and the outer surface of the waveguide may be in contact with a structure such as the Tenon's capsule, which has a refractive index close to 1.4. Light leakage to surrounding tissues could also be absorbed by the retina and/or the choroid as well as blood and other tissue fluids, causing potential phototoxicity. Therefore, to ensure high reflection at the outer surface and minimize optical loss to the external tissue, in some embodiments the reflectivity of the outer surface of the waveguide may be increased and/or the light loss from the outer surface may be decreased, for example by making the waveguide from a material having a higher refractive index than an adjacent layer or the surrounding tissue and/or by applying a reflective or light-blocking coating to the outside surface of the waveguide, i.e. the portion of the waveguide not facing the scleral tissue of the eyeball.

In one embodiment, the waveguide may be made of a high-refractive-index polymer having a refractive index greater than 1.5. For example, in one particular embodiment the waveguide may be constructed from a 1,3-glycerol dimethacrylate-based polymer, which has a refractive index close to 1.52 and which would have a critical angle of 65 deg. at the interface with a tissue having an index of refraction of 1.38. In other embodiments, the waveguide may be constructed from certain elastomers made of transparent styrenic copolymers having a refractive index of 1.577 and which give a critical angle of 61 deg. Waveguides such as these, made of high index of refraction materials, can efficiently guide excitation light coupled from low numerical-aperture pigtail fibers when the waveguides assume a curved geometry such as happens when the waveguides are wrapped around an eyeball in a subject. In various embodiments, a low refractive index polymer or hydrogel may be applied in a coating on the inner and/or outer surface of the high-index waveguide to further optimize the light delivery profile and possibly enhance biocompatibility.

In certain other embodiments, one or multiple light blocking layers may be applied at the outer surface of the waveguide to enhance internal reflection of light along the outer surface, and/or to reduce light loss from the outer surface of the waveguide to ensure that light is delivered primarily to the therapeutic side of the waveguide. To accomplish this, the waveguide may have a reflective or absorptive outer side that prevents light leakage and/or reflects light back into the waveguide core. However, for such a layer to be added, a core-cladded waveguide is necessary to maintain total internal reflection inside the waveguide that would be otherwise disturbed. In one embodiment, the absorptive layer may be a dye-doped polymer such as PDMS that could absorb any light that leaks from the waveguide core. In other embodiments, a thin sheet of reflective metal, such as aluminum or gold, may be applied to the outer surface of the waveguide. In such embodiments, an additional polymer layer may be deposited over the metal layer to prevent mechanical tissue prevent mechanical tissue damage that may otherwise arise from contact of the metal coating with tissue.

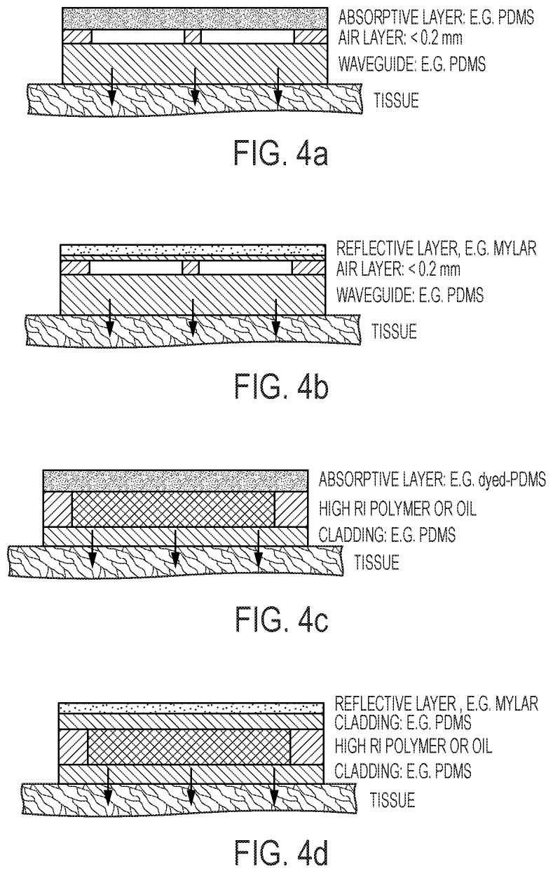

FIGS. 4a-4d show cross-sectional diagrams of core-cladded waveguides including various light-blocking and/or light-reflecting layers. In FIGS. 4a and 4b, an air spacer layer is incorporated into the waveguide to enable total internal reflection between air and the waveguide material (e.g. PDMS). In FIG. 4a, an absorptive layer (e.g. dyed-PDMS) is disposed over the air spacer layer to absorb any incidental light leakage from the waveguide. In another embodiment shown in FIG. 4b, a reflective sheet (e.g. Mylar) may be disposed over the air spacer layer of the waveguide; in some embodiments a layer of material such as PDMS may be disposed between the air spacer layer and the reflective sheet (FIG. 4b). In still other embodiments, the waveguide may be a core-cladded structure in which a high refractive index material core is surrounded by a lower index cladding (FIGS. 4c and 4d). The high refractive index core material may be one of various high refractive index polymers (such as polyurethane, 1,3-glycerol dimethacrylate-based polymer, transparent styrenic copolymers) or a high refractive index oil. The cladding layer adjacent the core may be absorptive (e.g. dyed PDMS, FIG. 4c) or non-absorptive, or an additional reflective layer (e.g. Mylar) may be disposed over the core and/or any cladding adjacent the core (FIG. 4d).

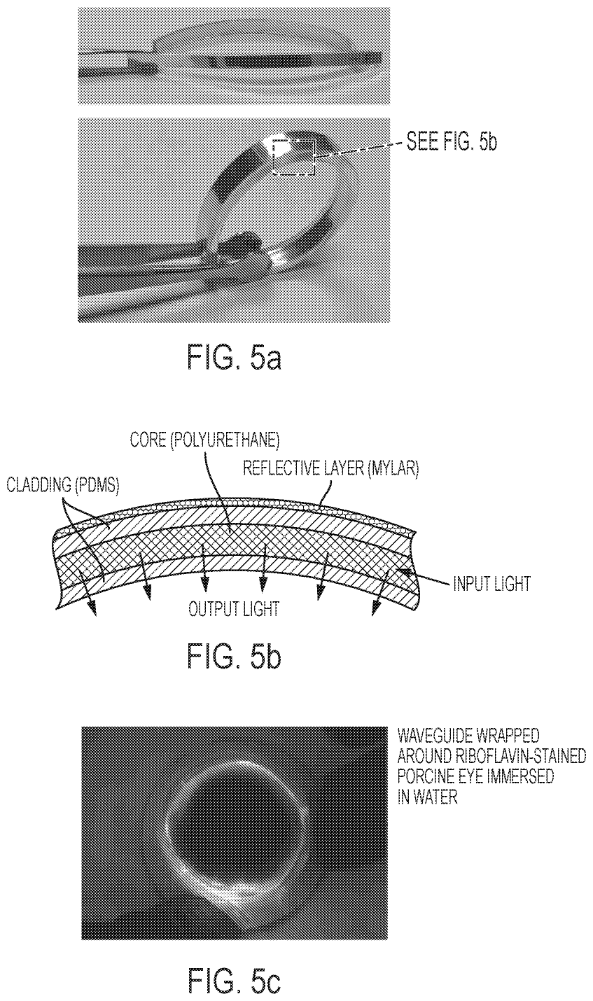

Based on the design of FIG. 4d, embodiments of a new flexible, polymer-based waveguide have been fabricated which include a core and cladding layers, where the core has a higher refractive index than the adjacent cladding. The cladding reduces light loss through surface reflections and enables light delivery irrespective of the refractive index of any material (e.g. adjacent tissue) that may surround the waveguide during use. The core-cladded waveguide structure also enables integration of a reflective sheet on one side of the waveguide without inhibiting total internal reflection inside the waveguide. One particular embodiment is shown in FIGS. 5a-5c. FIG. 5a shows photographs of the waveguide. FIG. 5b shows a cross-sectional diagram of a side view of the waveguide (see box in FIG. 5a) showing the layers as well as the flow of light through the waveguide. FIG. 5c shows a fluorescence image of the waveguide wrapped around an excised, riboflavin-stained porcine eyeball immersed in water. The innermost layer of the waveguide, the core (which may have a thickness≈1 mm), may be polyurethane, which has a refractive index ˜1.49 (FIG. 5b). Surrounding this layer on the top (i.e. the outer surface) and bottom (i.e. the inner surface) is a cladding layer of PDMS (thickness≈0.5 mm each), which has a refractive index˜1.41; the sides of the waveguide are also cladded with PDMS (e.g. 0.5-1 mm thickness; see FIG. 4d). On the outer surface of the waveguide the PDMS layer may have a reflective Mylar film (thickness<0.1 mm) placed thereon. Waveguides having such a design are flexible and deliver light only to one side of the waveguide (i.e. the inside), as shown diagrammatically in FIG. 5b. To simulate the in vivo situation, the waveguide was wrapped around the equator of a riboflavin-stained porcine eyeball and immersed in water. The light intensity emitted from the wave guide and delivered to the sclera was substantially uniform along the curved surface of the sclera, i.e. within a factor of 2 (or within about a 3 dB intensity range) as measured by the fluorescence intensity shown in FIG. 5c. Thus, in general, the disclosed waveguides provide substantially uniform delivery of light along the curved surface of a tissue, which facilitates providing relatively uniform light-related therapy to the curved tissue surface. Waveguides that have a tapered shape (e.g. having one end that is thicker than the other end and a transition of thickness in between the two ends, see below) can also provide substantially uniform light delivery along the surface of various tissues, including tissues that are relatively flat. For applications in which the waveguide will be placed adjacent a curved surface such as an eyeball, the waveguide may be made such that it has a curved shape that approximately matches the shape of the curved tissue surface with which it will be in contact (e.g. see FIG. 5a). In various embodiments the curved tissue surface may have a radius of curvature of 20 mm or less, 15 mm or less, 12 mm or less, or 10 mm or less.

Optimizing waveguide design to achieve uniform light extraction

To optimize waveguide design for uniform light extraction into tissues, the sources of waveguide loss and the distribution of light inside and extracted from the waveguide should be considered. Here, waveguide loss was modeled through surface scattering as a simple exponential decay along the length of the waveguide. Let Iᵢₙ (z) be the light intensity inside the waveguide at position z.

The change in light intensity inside the waveguide can expressed as:

$$\frac{{dI}_{i n}}{dz} = - ( γ + {α}_{scatt} ) * {I}_{i n} \qquad ( 2 )$$

Where γ is loss due to material absorption, and a_{scatt} is a scattering cross-section describing loss due to surface scattering. Given that a_{scatt}»γ for the waveguides, Equation 2 can be solved to obtain:

Iᵢₙ(z)=I₀*exp(−a_{scatt}z) (3)

Where I₀ (z) is the initial light intensity at z=0. The light extracted into tissues at position z can be expressed as:

$${I}_{out} = - \frac{{dI}_{i n}}{dz} = {α}_{scatt} * {I}_{0} = exp ( - {α}_{scatt} z ) \qquad ( 4 )$$

Thus, the extracted light profile is non-uniform with exponentially more light being extracted near the proximal end of the waveguide (near z=0 mm) compared to the distal end (near z=70 mm in one case). To compensate for this exponential attenuation, the waveguide can be designed by varying the scattering cross-section a_{scatt}, as a function of z. For uniform light delivery, the following is required:

$$\begin{matrix}\frac{{dI}_{out} ( z )}{dz} = 0 & ( 5 ) \\ \frac{d}{dz} ( \frac{{dI}_{i n}}{dz} ) = \frac{d}{dz} ( - {α}_{scatt} * {I}_{i n} ) = 0 & ( 6 ) \\ - \frac{d {α}_{scatt}}{dz} {I}_{i n} + {α}_{scatt}^{2} {I}_{i n} = 0 & ( 7 )\end{matrix}$$

Solving Equation 7, and assuming Iᵢₙ (z)≠0, the following is obtained:

$${α}_{scatt} ( z ) = \frac{1}{C - z} \qquad ( 8 )$$

Where C is a constant. To find an optimal a_{scatt} (z) for uniform light delivery, it is noted that a_{scatt}=A/t, where A is a constant, and t is the thickness of the waveguide. The physical interpretation of this equation is that, as the thickness of the waveguide decreases, there are more reflections inside the waveguide as a function of unit length and thus higher scattering loss. Assuming that a_{scatt} (z=0)=A/t₀, where t₀ is the initial waveguide thickness at z=0:

$$\begin{matrix}{α}_{scatt} ( z ) = \frac{A}{{t}_{0} - Az} = \frac{A}{t ( z )} & ( 9 ) \\ t ( z ) = {t}_{0} - Az & ( 10 )\end{matrix}$$

Equation 10 indicates that a tapered waveguide with decreasing thickness as a function of z is sufficient to obtain homogenous light extraction. In practice, a_{scatt} (z) can also be optimized by using different materials or coating layers to alter the scattering and refractive index profile of the waveguide, and to improve uniformity of light extraction.

To compute the extracted light profile with a tapered thickness t(z), Equation 2 needs to be rewritten as:

$$\frac{{dI}_{i n} ( z )}{dz} = - {α}_{scatt} ( z ) * {I}_{i n} ( z ) \qquad ( 11 )$$

Which has a solution of:

$${I}_{i n} ( z ) = {I}_{0} * exp [ - {\int }_{0}^{z} {α}_{scatt} ( z ) dz ] \qquad ( 12 )$$

The extracted light profile can be expressed as:

$${I}_{out} ( z ) = {I}_{0} * {α}_{scatt} ( z ) * exp [ - {\int }_{0}^{z} {α}_{scatt} ( z ) dz ] \qquad ( 13 )$$

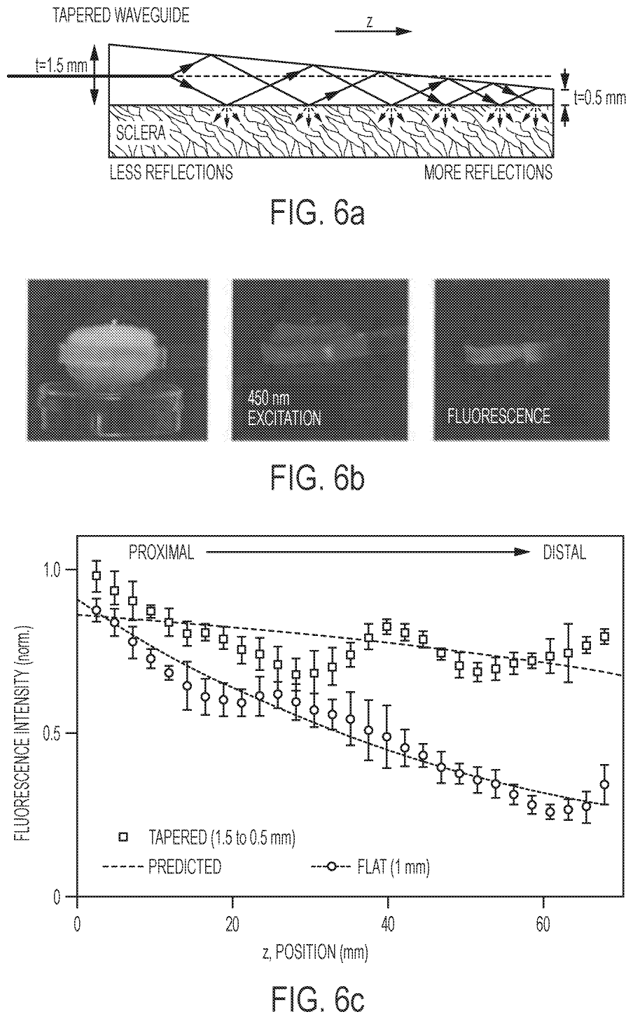

To validate the above theory, the light extraction profiles of flat and tapered waveguides were compared. As shown in the schematic of FIG. 6a, tapering the waveguide ensures less reflections towards the proximal end of the waveguide where the thickness is larger and more reflections towards the distal end where the thickness is smaller. This tapering compensates for the exponential attenuation that would otherwise be expected for a flat waveguide (see Equation 4).

To measure the uniformity of light extraction, 450 nm blue light was coupled into a waveguide wrapped around the equator of a riboflavin-stained porcine eyeball (FIG. 6b). The fluorescence intensity was measured around the eyeball for tapered waveguides (ranging from t=1.5 mm to t=0.5 mm) and flat waveguides with 1 mm thickness. Although the mean thickness of flat and tapered waveguides was the same (i.e. both about 1.0 mm), the variation of fluorescence intensity from proximal to distal was much greater for flat waveguides (FIG. 6c). The coefficient of variation (defined as standard deviation over mean) in fluorescence intensity was 36.3%, compared to just 10.7% for tapered waveguides.

To compare the results with theoretical considerations, the profile for flat 1 mm waveguides was fit to Equation 4, yielding a scattering loss coefficient of a_{scatt}=0.0175±0.0008 mm⁻¹. Using Equation 13, the predicted extraction profile is plotted in FIG. 4c for the tapered waveguides. Deviation from experimental data occurs since the model does not take into account the dependence of a_{scatt} on θ, the incident angle, and thus the numerical aperture of the coupling fiber. The heterogeneity of the scleral tissue which differs in thickness and refractive index along its circumference can also affect the estimation of a_{scatt} (z).

The results indicate significantly improved uniformity of light delivery using tapered waveguides as compared to flat waveguides. Using Equation 10, ideal linear tapering for uniform light extraction gives t(z)=t₀−0.0175z. For t₀=1.5 mm, t(z=70 mm)=0.28 mm. Thus, further improvement in uniformity may be achieved by increasing the tapering gradient of the waveguides or by optimizing other material properties. However, it should be noted that thin waveguides (<2 mm) are required for SXL due to anatomical constraints to scleral access in the orbit in vivo.

### Further Embodiments

In various embodiments, other synthetic polymers, such as poly(lactic-glycol acid), poly(ethylene glycol), and natural polymers, such as silk, may also be suitable materials for waveguide fabrication instead of PDMS. Waveguides made with materials with lower refractive indices than that of tissue, such as hydrogels, may be used for light delivery into tissues along a short distance. However, for certain embodiments of the SXL application it is preferred to use waveguides with indices higher than 1.38.

In particular embodiments, other crosslinking agents (besides Riboflavin) such as Rose Bengal can be applicable, which typically uses an excitation wavelength of 532 nm. In addition to SXL, the methods and systems disclosed herein may also be generally applicable to other light-activated therapies such as photodynamic therapy (exciting photosensitizers), photothermal therapies for cancer (for example, gold nanoparticles), ultraviolet therapy in dermatology, blue-light therapy for anti-microbial treatment, and low-level light therapy for pain relief and wound healing. In each of these other applications, the waveguide would be placed adjacent to the particular tissue (e.g. skin) and light having suitable properties (e.g. particular wavelengths and/or intensities) would be directed into the waveguide for suitable periods of time.

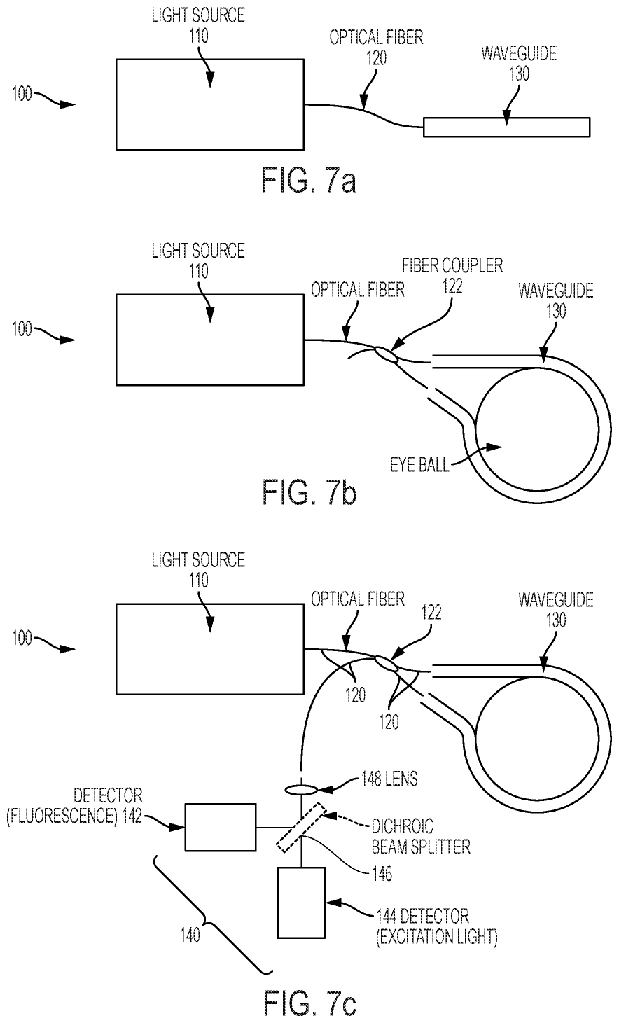

An embodiment of a light coupling system 100 is shown in FIG. 7a, in which a light source 110 is coupled to a waveguide 130 by an optical coupler, for example an optical fiber 120. In certain embodiments, the specific shape and dimension of the waveguide 130 may be tailored to match a desired geometry of the illumination area for particular applications. For SXL, the length of a waveguide 130 should generally be longer than 70 mm, the width larger than 3 mm, and the thickness less than 2 mm, in order to wrap around a typical subject's eyeball. The pigtail optical fiber may be attached to one end of the waveguide 130 as demonstrated above (FIG. 7a). However, in particular embodiments the optical coupler may include more than one optical fiber 120, such that a second optical fiber 120 may be attached to the other end of the waveguide 130 to improve uniformity of extracted light, and in one particular embodiment the waveguide 130 is tapered and in another embodiment the waveguide 130 is flat, i.e. has an approximately uniform thickness from end to end. Both pigtail lead fibers 120 may be two output ports of a 50/50 directional fiber-optic coupler 122 (or “fiber coupler”), which may be connected to the light source 110 (e.g. FIG. 7b). In certain embodiments, the waveguide 130 may be uniformly made of a single material, as demonstrated above, but may have a step core-cladding structure or graded index profile to achieve specific light extraction profiles. The light source 110 in various embodiments may be a laser or a light emitting diode (LED) which is selected to provide light at one or more wavelengths in a particular range of wavelengths, for example from 400 nm to 800 nm.

The optical system 100 in some embodiments may also include a monitoring setup or detector 140 to ensure appropriate light delivery (FIG. 7c). The detector 140 may include one or more of: a photodetector 142 to monitor fluorescence; a photodetector 144 to monitor excitation light; a dichroic beam splitter or dichroic filter 146; and/or a lens 148. For example, the output from the remaining port of the fiber-optic coupler 122, which may be directed through another optical fiber 120, can be directed to the photodetector 144 to measure the power of excitation light (e.g.450 nm) returning from the waveguide 130. The magnitude represents the amount of excitation light remaining in the waveguide 130 after propagating through the entire waveguide length and, therefore, it can serve as an indicator to ensure appropriate light coupling into the waveguide 130 from the light source 110 and whether there are any unexpected optical losses in the waveguide 130. The excited photochemical agent in the tissue—for example, riboflavin in SXL—generates fluorescence, part of which can be captured by the waveguide 130 and delivered to the ends of the waveguide 130 and then to the output port of the fiber-optic coupler 122. This fluorescence light (e.g. 500-600 nm for riboflavin) can also be measured by another photodetector 142, via the dichroic beam splitter or dichroic filter 146; in some embodiments the light may be focused onto the dichroic beam splitter or dichroic filter 146 by lens 148 (FIG. 7c).

Measurement of returning excitation and fluorescence light from the waveguide 130 could provide valuable information for clinicians to assist in treatment planning, dosimetry, and monitoring. For instance, sufficient photosensitizer staining can be verified prior to SXL, irradiation parameters (e.g. intensity, exposure time) can be tailored to specific patient characteristics (e.g. pigmentation, scattering and thickness of the sclera), and adjustments can be made in real-time.

Thus, in various embodiments, light from the light source 110 is directed to the waveguide 130 via the optical coupler, which may include one or more optical fibers 120 and/or the fiber-optic coupler 122. Light emitted from the waveguide 130 may be directed to a detector, which may include photodetector 142 to detect fluorescence light and/or another photodetector 144 to detect excitation light. The detector may include optical elements to shape and split the light emitted from the waveguide 130, for example a dichroic beam splitter or dichroic filter 146 and/or a lens 148.

In general the waveguide 130 has an elongated flat portion with a first end and a second end. The waveguide 130 may be tapered such that the first end is thicker than the second end. The thickness of the elongated flat portion of the waveguide 130 may be tapered from the first end to the second end in a linear or nonlinear fashion, tapering generally promoting a more uniform emission of light along the length of the waveguide 130. The elongated flat portion of the waveguide 130, when it contacts a tissue of a subject, may have an ‘inside’ (e.g. the side contacting the sclera when the waveguide 130 is wrapped around an eyeball) and an ‘outside’ (e.g. the side facing out, away from the sclera). In various embodiments a reflective coating may be applied to the ‘outside’ of the elongated flat portion in order to promote internal reflection of light, as disclosed herein. In some embodiments the subject is a human, although in other embodiments various animal subjects may be treated. The length and width of the elongated flat portion may be sufficient to wrap around an equatorial region of the eyeball or near the equatorial region. In various embodiments, the waveguide may be placed adjacent to at least a portion of the equatorial sclera, the posterior sclera, and/or the cornea of the eye of the subject.

In use, a tissue may be contacted by the waveguide 130 and light directed into the waveguide 130 by the light source 110. The light exits the waveguide 130 and interacts with a photosensitizer in the tissue; in the specific case of SXL treatment, riboflavin may be applied to the sclera and the interaction of blue light with the riboflavin leads to localized crosslinking and stiffening of the tissue. Light that is emitted from an end of the waveguide (e.g. fluorescence or excitation light) can be directed to a detector to help monitor the progress of a procedure such as SXL.

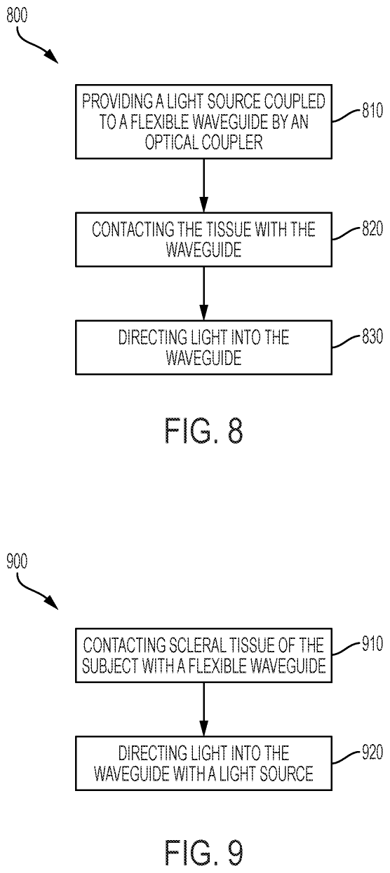

FIG. 8 is a flow chart of an example process 800 for delivering light to a tissue of a subject. The process 800 may include a step of providing a light source coupled to a flexible waveguide by an optical coupler (block 810). In some implementations, the waveguide may have a first end and a second end with an elongated flat portion therebetween, the first end having a first thickness and the second end having a second thickness, the first thickness being greater than the second thickness. The process 800 may further include a step of contacting the tissue with the waveguide (block 820). The process 800 may also include a step of directing light into the waveguide (block 830). The light may travel from the light source through the optical coupler into the waveguide, with at least a portion of the light exiting the waveguide toward the tissue.

FIG. 9 is a flow chart of an example process 900 of treating myopia in a subject. The process 900 may include a step of contacting scleral tissue of the subject with a flexible waveguide (block 910). The waveguide may have a first end and a second end with an elongated flat portion therebetween, with the first end having a first thickness and the second end having a second thickness, the first thickness being greater than the second thickness. The process 900 may also include a step of directing light into the waveguide with a light source (block 920). The light source may be coupled to the flexible waveguide by an optical coupler, with the light traveling from the light source through the optical coupler into the waveguide. At least a portion of the light may exit the waveguide toward the tissue and interact with a photosensitizer in the scleral tissue.

### Example—Periscleral Crosslinking of Ex Vivo Porcine Eyes

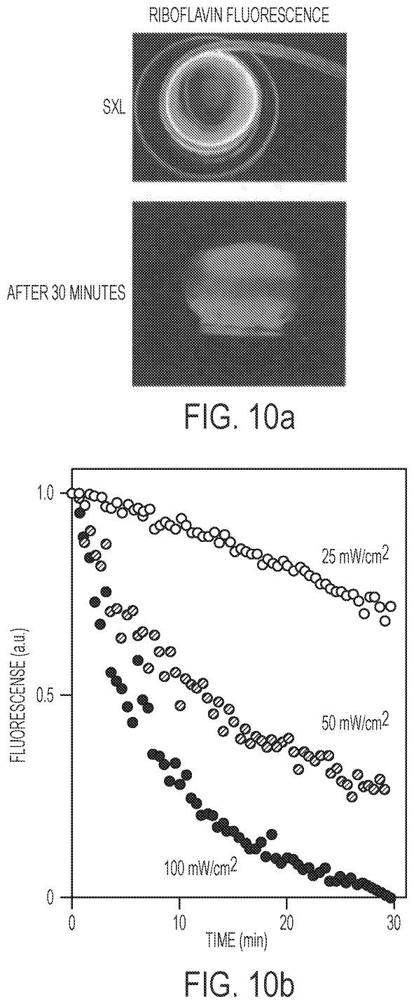

Using the tapered waveguides, SXL was conducted on fresh, excised porcine eyes with riboflavin and blue light (FIG. 10a). Porcine eyes were stained with 0.5% riboflavin and then exposed to 450 nm irradiation through the waveguides at mean intensities of 25-100 mW/cm² for 30 minutes, which is well below the reported threshold for tissue damage. The photobleaching of riboflavin was monitored during this process, and it was found that 50 mW/cm² was sufficient to bleach ˜75% of the riboflavin fluorescence (FIG. 10b).

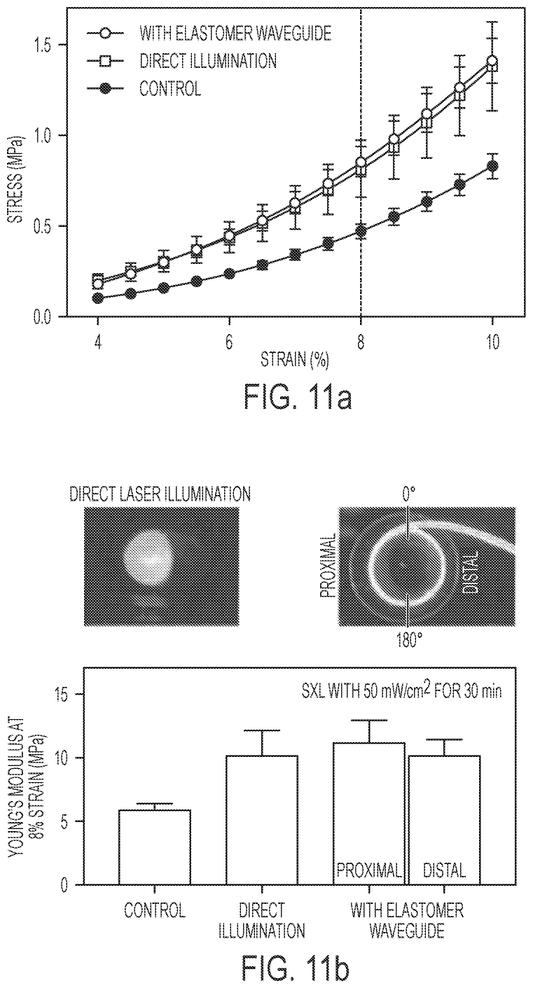

Following SXL, scleral stiffness was measured through conventional tensiometry. Stress-strain curves were obtained for scleral strips excised from eyes treated with SXL using the elastomer waveguide, direct illumination with a laser, and untreated eyes. FIGS. 11a and 11b show that there is no significant difference in stiffness when using the waveguide as compared to direct illumination at the same mean light intensities. The Young's modulus at 8% strain increased from 5.8±0.5 MPa for control eye to 10.2±1.9 MPa for eyes treated with direct illumination. For SXL with waveguides, sclera treated with the proximal half of the waveguide had a modulus of 11.2±1.7 MPa, and a modulus of 10.2±1.2 MPa in the distal half.

In both cases, SXL treatment resulted in a near 2-fold increase in the Young's modulus, which was statistically significant (p<0.001). With the elastomer waveguide, there was no significant difference in stiffness between the proximal and distally treated halves of the sclera (two tailed p=0.22). This results suggests that light extraction is sufficiently uniform for whole-globe sclera crosslinking.

The present invention has been described in terms of one or more preferred embodiments, and it should be appreciated that many equivalents, alternatives, variations, and modifications, aside from those expressly stated, are possible and within the scope of the invention.

1. A system for delivering light to a curved surface of a tissue of a subject, comprising:
a light source;
an optical coupler coupled to the light source; and
a flexible waveguide coupled to the optical coupler, the flexible waveguide having a first end and a second end with an elongated flat portion therebetween,
light from the light source being emitted substantially uniformly from along the elongated flat portion of the flexible waveguide, thereby delivering light substantially uniformly along the curved surface of the tissue.

2. The system of claim 1, wherein the flexible waveguide comprises a core having cladding attached to at least one side of the core.

3. The system of claim 2, wherein the core comprises a polymer or an oil having an index of refraction that is greater than an index of refraction of the cladding.

4. The system of claim 1, wherein the flexible waveguide comprises an absorptive layer or a reflective layer adjacent one side of the elongated flat portion.

5. The system of claim 4, further comprising an air gap between the flexible waveguide and the absorptive layer or the reflective layer.

6. The system of claim 1, wherein the optical coupler is coupled to the first end of the flexible waveguide.

7. The system of claim 1, wherein the optical coupler comprises a fiber coupler coupled to the light source and to the flexible waveguide.

8. The system of claim 7, wherein the optical coupler comprises a plurality of optical fibers coupled to the fiber coupler, wherein a first optical fiber of the plurality of optical fibers is coupled from the light source to the optical coupler and a second optical fiber of the plurality of optical fibers is coupled from the optical coupler to the first end of the flexible waveguide.

9. The system of claim 8, wherein a third optical fiber of the plurality of optical fibers is coupled from the optical coupler to the second end of the flexible waveguide.

10. The system of claim 9, wherein a fourth optical fiber of the plurality of optical fibers is coupled from the optical coupler to a light detector, the light detector detecting at least one of excitation light or fluorescence light from the fourth optical fiber.

11. The system of claim 1, wherein the first end of the flexible waveguide has a first thickness and the second end of the flexible waveguide has a second thickness, the first thickness being greater than the second thickness.

12. The system of claim 11, wherein the flexible waveguide is linearly tapered in thickness from the first end to the second end.

13. The system of claim 1, wherein the flexible waveguide is transparent in a wavelength range of 400 nm-800 nm.

14. The system of claim 1, wherein the flexible waveguide has an index of refraction of at least 1.5.

15. The system of claim 2, wherein the cladding comprises polydimethysiloxane (PDMS).

16. The system of claim 15, wherein the core comprises polyurethane.

17. The system of claim 16, wherein the reflective layer comprises Mylar.

18. The system of claim 1, wherein the curved surface of the tissue comprises at least a portion of an equatorial sclera, a posterior sclera, or a cornea of an eyeball of the subject, and wherein the elongated flat portion is placed adjacent the curved surface of the tissue of the subject.

19. The system of claim 1, wherein the flexible waveguide delivers light within a 3 dB range along the curved surface of the tissue.

20. The system of claim 1, wherein the curved surface of the tissue has a radius of curvature of 20 mm or less.

21-64. (canceled)
